(19) United States
(12) Patent Application Publication
(10) Pub. No.: US 2021/0395668 A1
(43) Pub. Date: Dec. 23, 2021

(54) Bioreactor Monitoring System using Spectrophotometer

(71) Applicant: Hypergiant Industries, Inc., Austin, TX (US)

(72) Inventors: Benjamin Edward Lamm, Dallas, TX (US); Jinseong Lee, Austin, TX (US); William R. Gavin, Lakeway, TX (US); Daniel David Haab, Austin, TX (US)

(21) Appl. No.: 17/354,804

(22) Filed: Jun. 22, 2021

Related U.S. Application Data
(60) Provisional application No. 63/042,965, filed on Jun. 23, 2020.

(51) Int. Cl. C12M 1/34; G01N 21/03; G01N 21/31; C12M 1/00; C12M 1/36

(52) U.S. Cl. CPC C12M 41/36; G01N 21/03; C12M 39/00; C12M 21/02; C12M 41/48; G01N 21/31

(57) ABSTRACT

A monitoring system for a bioreactor is disclosed. The monitoring system includes a cuvette arranged to receive a fluid from a bioreactor, wherein the fluid includes a portion of a biomass. The system further includes a light source arranged to project light, at wavelengths absorbable by the biomass, into the cuvette. A sensor is arranged to detect portions of the light that are not absorbed by the biomass. The system further includes a controller coupled to receive an indication of an amount of light detected by the sensor. The indication of the amount of detected light is usable to determine an amount of biomass in the cuvette.

### RELATED APPLICATIONS

This Application claims priority to U.S. Provisional Patent Application 63/042,965, file Jun. 23, 2020, and which is hereby incorporated by reference in its entirety.

### BACKGROUND

### Technical Field

The present disclosure relates generally to devices for producing biological organisms, and more particularly, the monitoring of growth of biological organisms in such devices.

### Description of the Related Art

Photobioreactors are reactors that utilize a light source to support the growth of phototrophic microorganisms in a controlled, artificial environment. Photobioreactors may be used to support photosynthetic growth of various different organisms using, e.g., carbon dioxide and light. Examples of organisms that have been grown using photobioreactors include algae (e.g., macroalgae and/or microalgae), plants, mosses, cyanobacteria, and purple bacteria.

### SUMMARY

A monitoring system for a bioreactor is disclosed. In one embodiment, a monitoring system includes a cuvette arranged to receive a fluid from a bioreactor, wherein the fluid includes a portion of a biomass. The system further includes a light source arranged to project light, at wavelengths absorbable by the biomass, into the cuvette. A sensor is arranged to detect portions of the light that are not absorbed by the biomass. The system further includes a controller coupled to receive an indication of an amount of light detected by the sensor. The indication of the amount of detected light is usable to determine an amount of biomass in the cuvette.

In one embodiment, the monitoring system may be used to determine an amount of biomass in the bioreactor. The controller may be coupled to a computer system, and may convey the indication (or information corresponding thereto) to the computer system. The computer system can then use this indication/information to determine an amount of biomass in the cuvette and thus extrapolate to determine the amount of biomass in the bioreactor. Over a number of samples, a rate of change of the amount of biomass in the bioreactor can also be determined.

In various embodiments, the light projected into the cuvette may be at wavelengths that are absorbable by the biomass. Thus, the sensor detects only the remaining portion of the light that is not absorbed by the biomass and thus passes through the cuvette. For example, the biomass may be one or more strains of algae, with light projected at wavelengths that are absorbed by those algae strains. The amount of light detected by the sensor may thus be less than the amount of light projected. Accordingly, the relationship between the amount of biomass in the cuvette and the amount of light detected by the sensor is an inverse relationship.

### BRIEF DESCRIPTION OF THE DRAWINGS

The following detailed description makes reference to the accompanying drawings, which are now briefly described.

FIG. 1 is a diagram of one embodiment of a monitoring system for monitoring an amount of biomass in a bioreactor.

FIG. 2 is a chart illustrating one embodiment of a sequence for cleaning a cuvette and performing a sample to determine an amount of biomass in a bioreactor.

FIG. 3 is a flow diagram illustrating one embodiment of a method for performing a sample to determine an amount of biomass in a bioreactor.

FIG. 4 is a flow diagram illustrating one embodiment for operating a monitoring system to sample biomass from a bioreactor and subsequently clean a cuvette.

FIG. 5 is a flow diagram illustrating one embodiment of a method for determining a rate of growth of a biomass in a bioreactor.

FIG. 6 is a drawing of one embodiment of a cuvette having a revolver structure.

Although the embodiments disclosed herein are susceptible to various modifications and alternative forms, specific embodiments are shown by way of example in the drawings and are described herein in detail. It should be understood, however, that drawings and detailed description thereto are not intended to limit the scope of the claims to the particular forms disclosed. On the contrary, this application is intended to cover all modifications, equivalents and alternatives falling within the spirit and scope of the disclosure of the present application as defined by the appended claims.

This disclosure includes references to “one embodiment,” “a particular embodiment,” “some embodiments,” “various embodiments,” or “an embodiment.” The appearances of the phrases “in one embodiment,” “in a particular embodiment,” “in some embodiments,” “in various embodiments,” or “in an embodiment” do not necessarily refer to the same embodiment. Particular features, structures, or characteristics may be combined in any suitable manner consistent with this disclosure.

Within this disclosure, different entities (which may variously be referred to as “units,” “mechanisms,” other components, etc.) may be described or claimed as “configured” to perform one or more tasks or operations. This formulation [entity] configured to [perform one or more tasks] is used herein to refer to structure (i.e., something physical), More specifically, this formulation is used to indicate that this structure is arranged to perform the one or more tasks during operation. A structure can be said to be “configured to” perform some task even if the structure is not currently being operated. A “controller configured to control a system” is intended to cover, for example, a controller that has circuitry that performs this function during operation, even if the controller in question is not currently being used (e.g., is not powered on). Thus, an entity described or recited as “configured to” perform some task refers to something physical, such as a device, circuit, memory storing program instructions executable to implement the task, etc. This phrase is not used herein to refer to something intangible.

Reciting in the appended claims that a structure is “configured to” perform one or more tasks is expressly intended not to invoke 35 U.S.C. § 112(f) for that claim element. Accordingly, none of the claims in this application as filed are intended to be interpreted as having means-plus-function elements. Should Applicant wish to invoke Section 112(f) during prosecution, it will recite claim elements using the “means for” [performing a function] construct.

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” or “responsive to” describes one or more factors that trigger an effect. This phrase does not foreclose the possibility that additional factors may affect or otherwise trigger the effect. That is, an effect may be solely in response to those factors, or may be in response to the specified factors as well as other, unspecified factors. Consider the phrase “perform A in response to B.” This phrase specifies that B is a factor that triggers the performance of A. This phrase does not foreclose that performing A may also be in response to some other factor, such as C. This phrase is also intended to cover an embodiment in which A is performed solely in response to B.

As used herein, the terms “first,” “second,” etc. are used as labels for nouns that they precede, and do not imply any type of ordering (e.g., spatial, temporal, logical, etc.), unless stated otherwise.

When used in the claims, the term “or” is used as an inclusive or and not as an exclusive or. For example, the phrase “at least one of x, y, or z” means any one of x, y, and z, as well as any combination thereof.

In the following description, numerous specific details are set forth to provide a thorough understanding of the disclosed embodiments. One having ordinary skill in the art, however, should recognize that aspects of disclosed embodiments might be practiced without these specific details. In some instances, well-known circuits, structures, signals, computer program instruction, and techniques have not been shown in detail to avoid obscuring the disclosed embodiments.

### DETAILED DESCRIPTION OF EMBODIMENTS

The present disclosure is directed to a monitoring system for a bioreactor. A bioreactor is an apparatus in which chemical processes that include the activity of biological organisms are carried out. For example, bioreactors can be used in the production of algae for other uses, although the disclosure is not limited to this application or this type of biomass.

In the apparatus of the present disclosure, the amount of biomass in the bioreactor may be periodically determined using the monitoring system. In particular, a sample of a fluid (e.g., water) that includes the biomass is transferred to a cuvette in the monitoring system. The cuvette includes optically clear (transparent) surfaces through which light can be projected, e.g., in the performance of spectrophotometry. The light projected into the cuvette may be done so at wavelengths that are absorbable by the biomass. A sensor receives a portion of the projected light that is not absorbed by the biomass and generates a corresponding indication. This indication is usable to determine the amount of biomass in the cuvette, which in turn may be extrapolated to determine the amount of biomass in the bioreactor. Over a number of periodic samples, a rate of change of the amount of biomass can be determined.

The monitoring system of the present disclosure also contemplates a cleaning cycle. Subsequent to performing a sampling, various embodiments of the monitoring system cause the cuvette to be flushed with a cleaning fluid. Embodiments are also possible and contemplated in which the cuvette includes a revolver-type structure wherein only one portion of the structure is actively used in the cuvette at a given time. Inactive portions may undergo a more thorough cleaning between uses as the active portion.

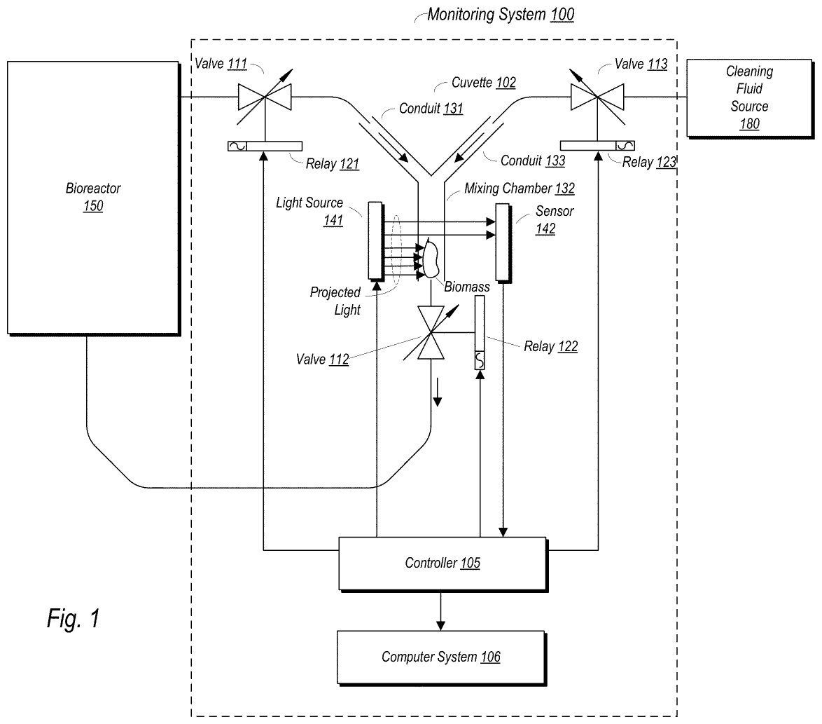

FIG. 1 is a diagram of one embodiment of a monitoring system for monitoring an amount of biomass in a bioreactor. In the embodiment shown, a monitoring system 100 is coupled to a bioreactor 150 which may be used for, among other things, growing biological organisms. The bioreactor 150 may contain a fluid in which the biological organisms are grown. For example, the bioreactor 150 may be used to grow one or more strains of algae, and may include one or more containers of water in which the algae are grown.

The monitoring system 100 shown here includes a cuvette 102 which is arranged to receive fluid from the bioreactor 150, as well as receiving fluid from a cleaning fluid source 180. A first valve 111 is coupled between the bioreactor 150 and a conduit 131 of the cuvette 102. The valve 111, in this embodiment, is controllable via a relay 121, which can be used to open and close the valve 111 based on signals received thereby. When open, the valve 111 allows fluid containing a portion of the biomass to flow from the bioreactor 150 into the conduit 131 of the cuvette 102.

The cuvette 102 of the illustrated embodiment includes both conduit 131 and conduit 133 which are commonly coupled to a mixing chamber 132. As noted above, a conduit 131 is arranged to convey fluid from the bioreactor 150 into the mixing chamber 132. A second conduit 133 is arranged to convey cleaning fluid into the mixing chamber 132. Similar to the arrangement discussed above, a valve 113 is coupled between the cleaning fluid source 180 and the cuvette 102. The valve 113 may be opened and closed by the relay 123 in response to a signal received by the latter.

Various types of cleaning fluid may be used for cleaning operations. In one embodiment, water is used as the cleaning fluid, although other types of fluids (which may include water) can be used as desired. Furthermore, in some embodiments, the cleaning fluid source 180 may be arranged to apply pressure to the cleaning fluid when conveyed into the cuvette 102. This may be useful, for example, when the types of biomass received by the cuvette 102 have a tendency to build up on its inner surfaces.

Another valve 112 and relay 122 combination in the embodiment shown is arranged to cause draining of the cuvette 102. The relay 122 may be used to open the valve 112 when it is desirable to drain fluid from the cuvette 102. When it is desired that the cuvette 102 retain fluid, the valve 112 is closed. After a sample of biomass is taken, the valve 112 may be opened to drain the cuvette 102 while returning the fluid containing the biomass back to the bioreactor 150. During cleaning operations, the mixing chamber 132 may be flushed with cleaning fluid while the valve 112 coupled thereto may remain open. Although not shown here, additional valves and pipes may be present to allow draining of the cleaning fluid to another location not associated with the bioreactor 150.

Sampling of the biomass may be performed using a light source 141 and a sensor 142 arranged near the mixing chamber 132 of the cuvette 102. The light source 141 may be any suitable type of light source, such as one or more light emitting diodes (LEDs), lasers (which can be coherent for specific wavelengths), or any other suitable type of illuminator. In various embodiments, the light source 141 projects light at wavelengths that are absorbable by the biomass. In one embodiment, the biomass comprises one or more strains of algae that absorb light in the visible spectrum range of 380-750 nanometers (nm). Accordingly, the light source 141 used in such an embodiment is selected to project light primarily within the visible spectrum. More generally, the wavelengths projected by the light source 141 in various embodiments may be selected based on the type of biomass to be processed in the bioreactor 150, and may include all or part of the visible spectrum.

Additional embodiments having different types of light sources are possible and contemplated. For example, an RGB (Red-Green-Blue) LED using pulse width modulation (PWM) is contemplated as a light source for another embodiment. The wavelengths used may be, in various embodiments, any visible wavelength in the 380-750 nm range. Generally, any suitable type of light source for the given implementation may be selected and used to project light into the cuvette for the purpose of sampling the biomass.

The sensor 142 is arranged to receive at least some of the light projected by the light source 141. In one embodiment, the sensor 142 may be arranged on an opposite side (relative to the light source 141) of the mixing chamber 132 of the cuvette 102. In some embodiments, the sensor 142 may be selected to be particularly sensitive to the wavelengths projected by the light source 141, while rejecting other wavelengths.

In the example of FIG. 1, an amount of biomass is present in the mixing chamber 132. The example further shows the light source 141 projecting light, as indicated by the arrows extending therefrom into, if not through, the cuvette 102. Some of the light in the example shown passes largely unobstructed through the mixing chamber 132 to the sensor 142. Other portions of the light are obstructed (e.g., absorbed) by the biomass. In practice, it is possible than some amount of light may pass through portions of the biomass (e.g., through gaps therein) to the sensor 142, although it is further possible that the intensity of such light may be attenuated. In various embodiments, the sensor 142 may determine an average of the intensity of detected light, with a corresponding indication generated based on the average. In other embodiments, sensor 142 may be arranged to detect light in both terms of intensity and distribution across the area thereof. With respect to the intensity, the sensor 142 of such an embodiment determines the intensity at which the light was detected. With respect to distribution, the sensor 142 in such an embodiment can determine variations in intensity of detected light, down to a total absence thereof when light is totally absorbed/blocked by the biomass in the mixing chamber 132. Generally speaking, sensor 142 may be any suitable sensor for detecting light in the given application of monitoring system 100.

Based on the detected light, the sensor 142 generates an indication, which may include one or more signals that are indicative of both the intensity and distribution of the light. In various embodiments, there is an inverse relationship between the amount of light detected by the sensor 142 (in terms of both intensity and distribution) and the amount of biomass in the mixing chamber.

Operations of the monitoring system 100 in the embodiment shown is conducted by a controller 105. In the embodiment shown, the controller 105 is coupled to the relays 121, 122, and 123 associated with valves 111, 112, and 113, respectively. Controller 105 is also coupled to the light source 141, and the sensor 142. To cause a sample of the biomass to be conducted, the controller 105 may cause the relay 121 to open valve 111, while keeping valves 112 and 113 shut. The valve 111 may be held open for a specified amount of time and/or until the cuvette 102 has received a specified amount of fluid (containing biomass) from bioreactor 150. Upon the cuvette 102 having received the specified amount of fluid, controller 105 may change the state of signals conveyed to the relay 121 to cause the valve 111 to be shut.

After allowing some settling time following the transfer of fluid from the bioreactor 150 into the cuvette 102, controller 105 continues the sampling process by conveying a signal to light source 141. Responsive to this signal, light source 141 begins projecting light into the cuvette 102. If biomass is present in the portion of cuvette 102 in which the light is projected (e.g., mixing chamber 132 in this example), some of this light may be absorbed. As noted above, light source 141 is configured in various embodiments to project light at wavelengths that are absorbed by the expected type of biomass. The light that traverses the entire distance through cuvette 102 may be detected by sensor 142. The light detected by sensor 142 results in the generation of an indication that is usable to determine the amount of biomass currently in the cuvette 102. The indication in one embodiment comprises signals indicative of the intensity and distribution of light received by the sensor.

Monitoring system 100 also includes computer system 106. Controller 105 in the embodiment shown forwards the indication received from sensor 142 to computer system 106. Embodiments are possible and contemplated in which controller 105 performs some pre-processing on the indication prior to forwarding to computer system 106. Upon receiving the indication (pre-processed or not), computer system 106 determines the amount of biomass present in the cuvette 102 for the given sample. Computer system 106 may also extrapolate from the amount of biomass determined to be in cuvette 102 to calculate an amount of biomass present in bioreactor 150. Over a number of different samples, computer system 106 may determine a rate of change (e.g., rate of growth) of the amount of biomass in the bioreactor. For example, if the bioreactor 150 is used to generate algae, computer system 106 may determine the rate of growth of the algae, and this information can be used to determine a time to harvest.

Upon completing of a sample in the illustrated embodiment, the controller 105 conveys a signal to relay 122 in order to cause the opening of the valve 112. When valve 112 is opened, the cuvette 102 is drained of fluids contained therein. In this example, the fluid, including the biomass is transferred back to the bioreactor 150. Although not explicitly shown, a pump may be implemented between valve 112 and bioreactor 150 to facilitate the transfer of fluid.

A cleaning operation may be performed by monitoring system subsequent to conducting a sample. After the fluid containing the biomass has been transferred back into the bioreactor 150, controller 105 may initiate the cleaning operation by causing relay 123 to open valve 113. When open, valve 113 allows cleaning fluid to be transferred into conduit 133 of cuvette 102. In some embodiments, cleaning fluid source 180 may include a pump or other mechanism to cause the cleaning fluid to be provided at a pressure greater than ambient pressure. The cleaning fluid may be any suitable cleaning fluid, and may be as simple as water. The cleaning fluid may exit cuvette 102 through the bottom of mixing chamber 132 and through valve 112, which remains open during the cleaning operation. In some embodiments, the cleaning fluid (e.g., water) may be transferred to bioreactor 150 and can thus be used as a base for the growth of biomass therein. In other embodiments, additional valves not explicitly shown here may be present to drain the cleaning fluid away from the bioreactor. The cleaning operation may be terminated by controller 105 causing relay 123 close valve 113 and, subsequently (e.g., after the cuvette has been fully drained), causing relay 122 to close valve 112.

In various embodiments, the controller functions carried out by controller 105 may be performed using artificial intelligence (AI) based analysis. For example, machine learning can be used to account for cloudiness in performing samples, where the cloudiness results from biomass adhering to the inner surfaces of cuvette 102 even after cleaning. Various forms of AI-based analysis may also be used to, e.g., adjust the sampling periodicity or other functions related to the automation of the sampling process. The AI/machine learning functions may be carried out on computer system 106 in one embodiment. In another possible embodiment, controller 105 may include functionality capable of carrying out the various AI/machine learning functions. These functions may also be divided between controller 105 and computer system 106 in some embodiments.

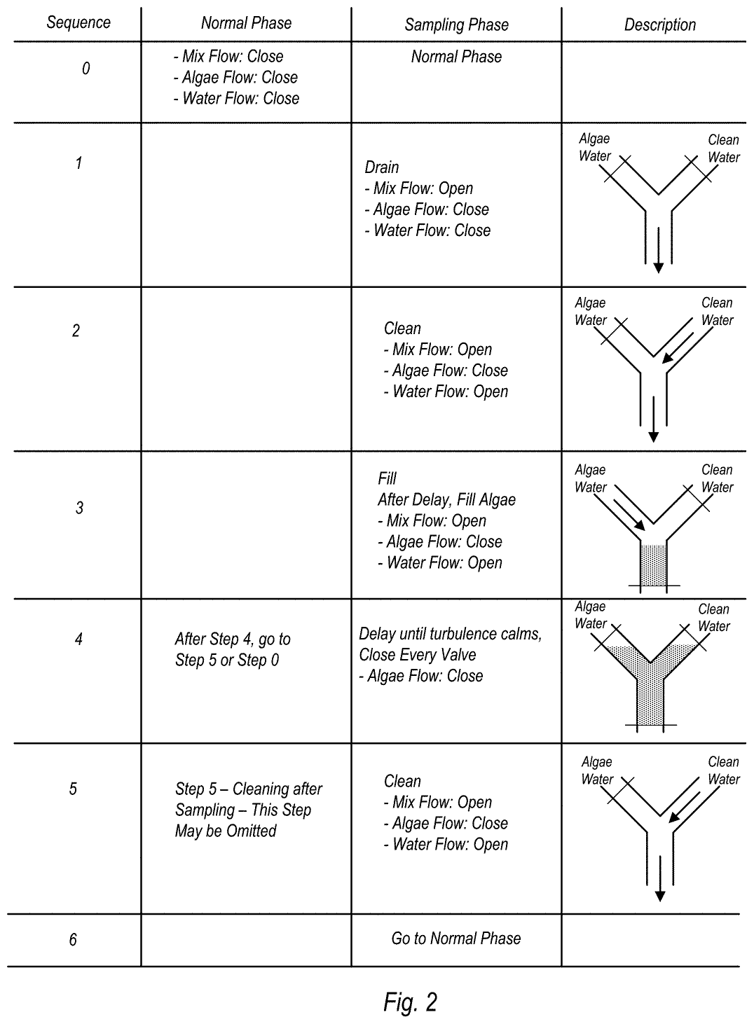

The sequence discussed above is further illustrated in the table illustrated in FIG. 2. For the purposes of discussion here, the mix flow valve corresponds to valve 112 of FIG. 1, which is coupled to the mixing chamber 132 of cuvette 102. The algae flow valve corresponds to valve 111 of FIG. 1, which is coupled between bioreactor 150 and conduit 131 of cuvette 102 (and thus, this example corresponds to one in which algae is grown in bioreactor 150). The water flow valve corresponds to valve 113 of FIG. 1, which is coupled between cleaning fluid source 180 and conduit 133 of cuvette 102.

It is noted that the sequence numbers shown in FIG. 2 do not necessarily correspond to the order of operations that occur during operation of various embodiments of monitoring system 100. In contrast, the order of operations may vary. Instead, the chart of FIG. 2 is provided to illustrate the various operations that are carried out by some embodiments of the monitoring system 100 of FIG. 1.

In sequence step 0 in the chart of FIG. 2, the mix flow, algae flow, and water flow valves are all closed. This is referred to as the normal phase, and cuvette 102 may be substantially empty at this time.

In sequence step 1, the mix flow valve is open while the algae flow and water flow valve remains closed. If any excess fluid is still present in cuvette 102 at this time, it may be drained in response to the opening of the mix flow valve. Sequence step 2 corresponds to the cleaning operation, and includes both the mix flow valves and water flow valves being open. It is noted that the cleaning operation may be performed both before and after a sampling operation in some embodiments.

A fill operation is illustrated as sequence step 3. During the fill operation, the algae flow valve is open, while the water flow and mix flow valves are closed. There may, in some embodiments, be a delay (e.g., 10 seconds, as shown here) between the time the valves are positioned as listed and the beginning of algae water flow into the cuvette 102. Once flow begins, algae water flows into cuvette 102 through conduit 131. The fill operation may continue for a specified time and/or until a specified amount of algae water has been transferred into the cuvette 102.

In sequence step 4, a sampling operation is performed. Upon filling the cuvette 102 to the specified amount, the algae flow valve is closed. After the fill operation is complete, there may be a waiting period (e.g., 5 seconds as shown here) to allow the settling of the fluid transferred. When enough time has elapsed for the fluid to settle, sampling may be performed. In this particular example, sampling includes activating a red LED, reading the sensor, and taking an average of the light value detected thereby. This average may be used to generate an indication that is forwarded to, e.g., controller 105 and computer system 106 for further processing to determine an amount of algae present in the cuvette during the given sample. It is noted, however, that the light source may be something other than an LED, and similarly, the sensor may operate in a different manner than in this particular example.

In sequence step 5, a cleaning operation (similar to that of sequence step 2) may be performed. In some embodiments, the cleaning operation may be skipped at this point. Generally speaking, a cleaning operation may be performed at any desired time in accordance with the specifications of the particular implementation of monitoring system 100. In the example shown, after completion of sequence step 5, and in accordance with sequence step 6, the monitoring system 100 may return to the normal phase.

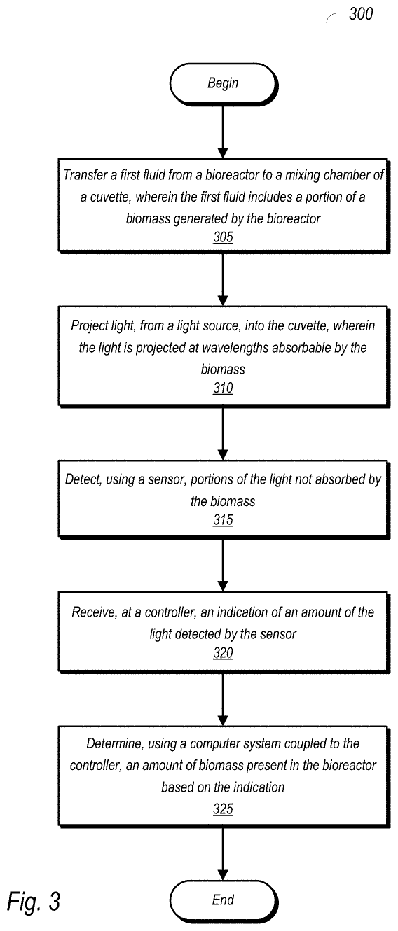

FIG. 3 is a flow diagram illustrating one embodiment of a method for performing a sample to determine an amount of biomass in a bioreactor. Method 300 of FIG. 3 may be performed with various embodiments of a bioreactor monitoring system, including (but not limited to) that shown in FIG. 1. Embodiments of a monitoring system not explicitly discussed herein but otherwise capable of carrying out Method 300 may thus fall within the scope of this disclosure.

Method 300 includes transferring a first fluid from a bioreactor to a mixing chamber of a cuvette, wherein the first fluid includes a portion of a biomass generated by the bioreactor (block 305). The method further includes projecting light, from a light source, into the cuvette, wherein the light is projected at wavelengths absorbable by the biomass (block 310). After projecting the light from the light source, the method includes detecting, using a sensor, portions of the light not absorbed by the biomass (block 315). After detecting portions of the light not absorbed by the biomass, the method continues with receiving, at a controller, an indication of an amount of the light detected by the sensor (block 320). Thereafter, the method includes determining, using a computer system coupled to the controller, an amount of biomass present in the bioreactor based on the indication (block 325).

In various embodiments, the method includes, repeating, over a number of periodic intervals, the transferring, the projecting, the detecting, the receiving and the determining. Based on these repeated samples, the method may also include calculating, using indications received over the number of periodic intervals a rate of growth of the biomass.

Performing a sample in various embodiments includes causing a first relay to open a first valve using the controller and receiving, in the cuvette, the first fluid from the bioreactor, in response to opening the first valve. Thereafter, the method includes causing the first relay to close the first valve subsequent to the cuvette receiving a specified amount of the first fluid.

Cleaning operations may also be performed at various times during operation of the monitoring system. Accordingly, the method further includes the controller causing a cleaning operation to be performed, wherein performing the cleaning operation comprises. Causing the cleaning operation includes the controller causing a second relay to open a second valve. In response to opening the second valve, the method includes draining the cuvette in response to opening the second valve and causing a third relay to open a third valve, using the controller. Thereafter, the method includes flushing the cuvette with a cleaning fluid received via the third valve. To complete the clearing operation, the flow of cleaning fluid may be cut off by closing the third valve after a specified amount of time, followed by closing the second valve subsequent to closing the third valve.

The monitored biomass may be virtually any type of biomass that can be part of a biochemical reaction occurring in a bioreactor. In one embodiment, the biomass may be one or more different strains of algae. In such an embodiment, projecting light at wavelengths absorbable by the biomass comprises projecting light at wavelengths from 380 nanometers to 750 nanometers, which is the visible spectrum.

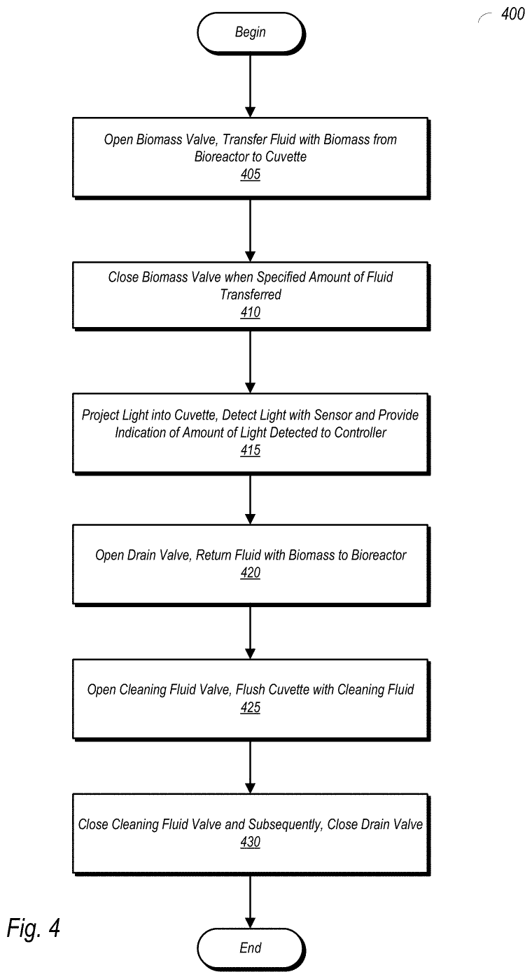

FIG. 4 is a flow diagram illustrating one embodiment for operating a monitoring system to sample biomass from a bioreactor and subsequently clean a cuvette. Method 400 as shown here may be performed by various embodiments of the monitoring system discussed above. Other embodiments of a monitoring system not explicitly discussed herein that are capable of carrying out Method 400 may also fall within the scope of this disclosure.

Method 400 includes opening a biomass valve and transferring fluid containing a portion of the biomass therein into a cuvette (block 405). The biomass valve may correspond to, for example, valve 111 of monitoring system 100 shown in FIG. 1. After a specified amount of the biomass-containing fluid has been transferred, the biomass valve is closed (block 410). In one embodiment, the amount of biomass-containing fluid that has been transferred may be determined by transferring the fluid for a specified amount of time at a particular flow rate.

Upon completing the transfer of the biomass-containing fluid, the fluid may be allowed to settle for a specified time. Once settle, the method continues with the projecting of light into the cuvette, the detecting of an amount of light passing through the cuvette, generating an indication of the amount of light detected, and providing the indication to a controller (block 415). The light may, in various embodiments, be projected at wavelengths that are absorbable by the particular type of biomass. The amount of light detected may thus have an inverse relationship with the amount of biomass in the cuvette. Thus, greater amounts of biomass present in the cuvette may correspond to lower amounts of light detected.

After the indication has been generated, Method 400 continues with the opening of a drain valve and the return of the biomass-containing fluid to the bioreactor (block 420). The drain valve may correspond to, e.g., valve 112 of the system illustrated in FIG. 1. In the illustrated embodiment, a cleaning operation is performed subsequent to the sampling. Accordingly, Method 400 further includes opening a cleaning fluid valve and flushing the cuvette with cleaning fluid (block 425). The cleaning fluid may be any suitable fluid, including water. The cleaning fluid valve may, in one embodiment, correspond to valve 113 of the system illustrated in FIG. 1. Upon completing the cleaning, the fluid valve may be closed to discontinue flushing the cuvette, followed by closing of the drain valve (block 430).

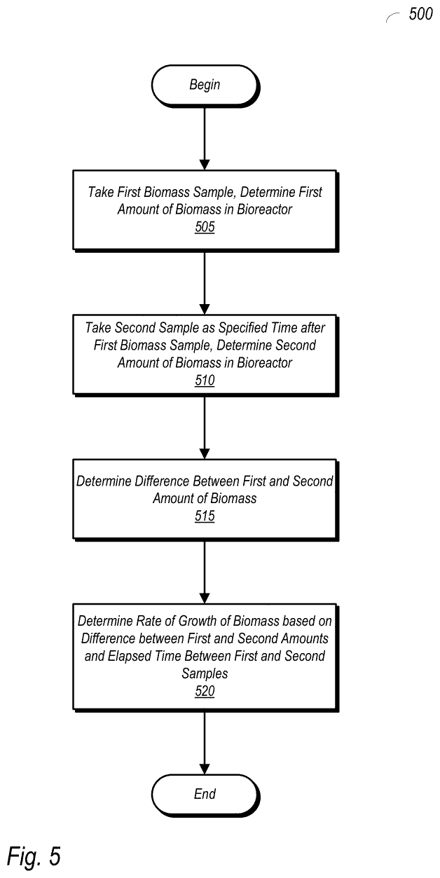

FIG. 5 is a flow diagram illustrating one embodiment of a method for determining a rate of growth of a biomass in a bioreactor. Method 500 may be performed using various embodiments of the monitoring system 100 discussed above. Embodiments of a monitoring system capable of carrying out Method 500 but not explicitly discussed herein may nevertheless fall within the scope of this disclosure.

Method 500 begins with the taking of a first sample of biomass to determine a first amount of biomass in a bioreactor (block 505). The sample may determine an amount of biomass in a cuvette. Subsequently, an extrapolation may be performed to determine an amount of biomass in the bioreactor based on the amount of biomass detected in the cuvette. At a specified time subsequent to that of the first biomass sample, a second sample is taken to determine a second amount of biomass in the bioreactor (block 510). A difference between the first and second amounts of biomass is then determined (block 515). Based on the elapsed time and difference in amount of biomass between the first and second sample, the rate of growth of the biomass is determined (block 520).

More generally, the methodology discussed herein contemplates determining a rate of change of the amount of biomass. The change in the biomass can include an increase or a decrease. The information obtained by this methodology can be used in various ways. For example, the information may be used to determine when it is time to harvest the biomass when the bioreactor is used in a production environment. In another example, the methodology discussed herein may be used to determine the efficacy of a process used to generate biomass in, e.g., a research environment.

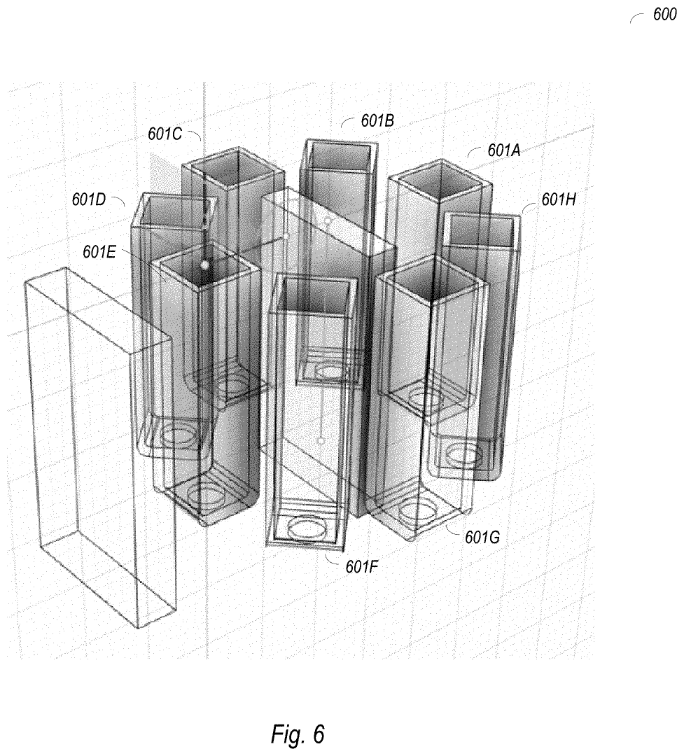

FIG. 6 is a drawing illustrating one embodiment of a revolver structure that can be used as part of a cuvette in accordance with this disclosure. In the illustrated embodiment, revolver structure 600 includes eight different conduits, 601A-601H. These conduits may be rotated in the revolver structure 600 such that one of them is an active part of the cuvette at a given time. For example, conduit 601A may be rotated into the structure such that it becomes the active mixing chamber of cuvette 102 as discussed above. Conduits 601B-601H may remain inactive. At a subsequent time, the revolver structure may be rotated such that, e.g., conduit 601B is the active conduit, while conduits 601A and 601C-601H are inactive. The schedule for rotation may vary from one embodiment to the next, with any suitable or desired schedule used.

Although the example above is applied to the mixing chamber portion of cuvette 102, the revolver structure may be utilized with, e.g., either one or both of the conduits 131 and 133. Embodiments are possible and contemplated in which all three portions of the cuvette shown in FIG. 1 utilize an embodiment of a revolver structure 600. Although not explicitly shown, embodiments of the revolver structure 600 may include, e.g., gaskets or other sealing mechanisms to ensure a good seal between the components, thereby preventing leakage during the transfer of fluids.

Utilization of an embodiment of the revolver structure 600 may allow for more thorough cleaning of inactive portions thereof. For example, manual cleaning may be performed on portions of the revolver structure not currently in use and/or may allow for the use of stronger cleaning agents while alleviating the chances of contaminating the biomass/fluid in the bioreactor.

Numerous variations and modifications will become apparent to those skilled in the art once the above disclosure is fully appreciated. It is intended that the following claims be interpreted to embrace all such variations and modifications.

What is claimed is:

1. An apparatus, comprising:
a monitoring system configured to determine an amount of biomass in a bioreactor at a given time, wherein the monitoring system includes:
a cuvette configured to receive fluid from the bioreactor, the fluid including a portion of the biomass;
a light source arranged to project light, at wavelengths absorbable by the biomass, into the cuvette;
a sensor arranged to detect portions of the light not absorbed by the biomass; and
a controller coupled to receive an indication of an amount of the light detected by the sensor, wherein the indication is usable to detect an amount of biomass in the cuvette.

2. The apparatus of claim 1, wherein the cuvette includes a first conduit configured to receive the fluid from the bioreactor, a second conduit configured to receive a cleaning fluid, and a mixing chamber coupled to the first and second conduits.

3. The apparatus of claim 2, further comprising:
a first valve coupled the first conduit;
a second valve coupled to the mixing chamber,
a third valve coupled to the second conduit; and
first, second and third relays configured to operate the first, second, and third valves, respectively, wherein the first, second and third relays are controllable by the controller.

4. The apparatus of claim 3, wherein the controller is configured to cause performing of a sampling process, wherein, in causing performance of the sampling process, the controller is configured to:
cause the cuvette to receive fluid containing the biomass by activating a signal provided to the first relay, wherein the first valve is opened in response to activating the signal provided to the first relay; and
cause the first valve to be closed by deactivating the signal provided to the first relay in response to the cuvette receiving a specified amount of the fluid containing the biomass.

5. The apparatus of claim 3, wherein the controller is configured to:
cause the cuvette to be drained by activating a signal provided to the second relay, wherein the second valve is opened in response to activating the signal provided to the second relay; and
cause the third valve to be closed by deactivating the third signal provided to the third relay.

6. The apparatus of claim 3, wherein the controller is configured to cause a cleaning process to be performed on the cuvette, wherein, in performing the cleaning process, the controller is configured to:
cause the cuvette to receive the cleaning fluid by activating a signal provided to the third relay, wherein the third valve is opened in response to activating the signal provided to the third relay and subsequent to causing the relay to open the second valve;
cause the third valve to be closed by deactivating the signal provided to the third relay in response after a specified amount of time has been elapsed; and
cause the second valve to be closed subsequent to closing the third valve.

7. The apparatus of claim 2, wherein the cuvette comprises a revolver structure including a plurality of mixing chambers, wherein the revolver structure is rotatable to move one of the plurality of mixing chambers into contact with the first and second conduits such that the one of the plurality of mixing chambers is an active mixing chamber, and wherein remaining ones of the plurality of mixing chambers are inactive when the one of the plurality of mixing chambers is the active mixing chamber.

8. The apparatus of claim 1, wherein the light source is configured to project light at wavelengths between 380 and 750 nanometers.

9. The apparatus of claim 1, wherein the controller is configured to periodically cause sampling of the amount of biomass in a mixing chamber, wherein the sampling comprises the controller:
causing biomass to be provided from the bioreactor into the mixing chamber;
causing the light source to project light into the mixing chamber; and
receive the indication of an amount of the light detected by the sensor.

10. The apparatus of claim 1, further comprising a computer system coupled to the controller, wherein the computer system is configured to:
determine, for a given sample, an amount of biomass in a mixing chamber based on the indication; and
determine, for a plurality of successive samples, a rate of growth of the biomass in the bioreactor.

11. A method comprising:
transferring a first fluid from a bioreactor to a mixing chamber of a cuvette, wherein the first fluid includes a portion of a biomass generated by the bioreactor;
projecting light, from a light source, into the cuvette, wherein the light is projected at wavelengths absorbable by the biomass;
detecting, using a sensor, portions of the light not absorbed by the biomass;
receiving, at a controller, an indication of an amount of the light detected by the sensor; and
determining, using a computer system coupled to the controller, an amount of biomass present in the bioreactor based on the indication.

12. The method of claim 11, further comprising:
repeating, over a number of periodic intervals, the transferring, the projecting, the detecting, the receiving and the determining; and
calculating, using indications received over the number of periodic intervals a rate of growth of the biomass.

13. The method of claim 11, further comprising:
causing a first relay to open a first valve using the controller;
receiving, in the cuvette, the first fluid from the bioreactor, in response to opening the first valve; and
causing the first relay to close the first valve subsequent to the cuvette receiving a specified amount of the first fluid.

14. The method of claim 13, further comprising the controller causing a cleaning operation to be performed, wherein performing the cleaning operation comprises:
causing a second relay to open a second valve, using the controller;
draining the cuvette in response to opening the second valve;
causing a third relay to open a third valve, using the controller;
flushing the cuvette with a cleaning fluid received via the third valve;
closing the third valve after a specified amount of time; and
closing the second valve subsequent to closing the third valve.

15. The method of claim 11, wherein projecting light at wavelengths absorbable by the biomass comprises projecting light at wavelengths from 380 nanometers to 750 nanometers, and wherein the biomass is algae.

16. A system comprising:
a cuvette having a first conduit, a second conduit, and a mixing chamber coupled to the first and second conduits;
a first valve configured to, when open, cause a first fluid to be provided to the mixing chamber via first conduit, the first fluid including a portion of a biomass generated by a bioreactor;
a second valve configured to, when open, cause the mixing chamber to be drained;
a third valve configured to, when open, cause a second fluid to be provided to the mixing chamber via the second conduit, the second fluid being a cleaning fluid;
a light source arranged to project light, at one or more wavelengths absorbable by the biomass, into the mixing chamber;
a sensor arranged to receive a portion of the light; and
a controller configured to cause, when at least the mixing chamber is full with the first fluid, the light source to project the light and further configured to receive an indication of an amount of light received by the sensor, wherein the indication is usable to determine an amount of biomass present in the bioreactor.

17. The system of claim 16, further comprising:
a first relay configured to operate the first valve in response to a first signal received from the controller;
a second relay configured to operate the second valve in response to a second signal received from the controller; and
a third relay configured to operate the third valve in response to a third signal received from the controller.

18. The system of claim 16, wherein the controller is configured to cause periodic samples to be taken to determine the amount of biomass in the bioreactor, wherein in performing the periodic samples, the controller is configured to:
cause the first valve to be opened for a duration to allow the mixing chamber to fill with the first fluid;
cause the light source to project the light into the mixing chamber;
receive the indication from the sensor; and
forward the indication to a computer system, wherein the computer system is configured to determine the amount of biomass in the bioreactor based on the indication.

19. The system of claim 18, wherein the computer system is configured to determine a rate of growth of the biomass over a number of periodic samples.

20. The system of claim 18, wherein the controller is configured to cause a cleaning cycle to be performance subsequent to ones of the periodic samples, wherein, during performance of the cleaning cycle, the controller is configured to:
cause the second valve to open; and
cause the third valve to open, wherein the mixing chamber is flushed with the second fluid in response to opening the third valve.
